(19) United States
(12) Patent Application Publication
(10) Pub. No.: US 2007/0043414 A1
(43) Pub. Date: Feb. 22, 2007

(54) Lead fixation and extraction

(76) Inventors: Daniel W. Fifer, Windsor, CA (US); Terrance Ransbury, Chapel Hill, NC (US)

Correspondence Address: STALLMAN & POLLOCK LLP, 353 SACRAMENTO STREET, SUITE 2200, SAN FRANCISCO, CA, 94111, US

(21) Appl. No.: 11/504,383

(22) Filed: Aug. 15, 2006

Related U.S. Application Data
(60) Provisional application No. 60/708,143, filed on Aug. 15, 2005.

(51) Int. Cl. A61N 1/05

(52) U.S. Cl. 607/126

(57) ABSTRACT

A device for implantation in the vasculature of a patient can include a fixation mechanism for anchoring the device in place while allowing for easy removal of the device. The fixation mechanism can include a detachable and/or biodegradable portion that can allow for removal from the bulk of the device in order to allow for the bulk to simply be pulled from the body without likelihood of injury. These devices also can include electrode assemblies that do not promote fibrous in growth, further reducing the likelihood for injury upon extraction of the device.

The present application claims priority to U.S. Provisional Application No. 60/708,143 filed Aug. 15, 2005.

### BACKGROUND

There are a number of medical devices that can have portions implanted into a patient's vasculature. For example, pacemakers and implantable cardioverter-defibrillator (ICDs) systems (i.e. devices with leads) have been successfully implanted for years for treatment of heart rhythm conditions. Pacemakers are implanted to detect periods of bradycardia and deliver electrical stimuli to increase the heartbeat to an appropriate rate, while ICDs are implanted in patients to cardiovert or defibrillate the heart by delivering electrical current directly to the heart. Another implantable defibrillation device can detect an atrial fibrillation (AF) episode and deliver an electrical shock to the atria to restore electrical coordination.

Next generation ICDs, pacemakers, etc., may take the form of elongated intravascular devices, such as those described, for example, in U.S. Pat No. 7,082,336, entitled “IMPLANTABLE INTRAVASCULAR DEVICE FOR DEFIBRILLATION AND/OR PACING,” filed Jun. 4, 2003; U.S. patent application Ser. No. 10/453,971, entitled “DEVICE & METHOD FOR RETAINING A MEDICAL DEVICE WITHIN A VESSEL”, filed Jun. 4, 2003; as well as U.S. patent application Ser. No. 10/862,113, entitled “INTRAVASCULAR ELECTROPHYSIOLOGICAL SYSTEM AND METHODS,” filed Jun. 4, 2004, each of which is hereby incorporated herein by reference. Such a device can be implanted in a number of alternative ways, including methods described in U.S. patent application Ser. No. 10/862,113, filed Jun. 4, 2004, incorporated by reference above. For example, the device can be introduced into the venous system via the femoral vein, introduced into the venous system via that subclavian vein or the brachiocephalic veins, or into the arterial system using access through one of the femoral arteries. Moreover, different components of the intravascular systems may be introduced through different access sites. For example, a device may be separately introduced through the femoral vein and a corresponding lead may be introduced via the subclavian vein.

The chronic implantation of a lead for one of these devices, or for more conventional devices, in a ventricle, great cardiac vein, or other similar location inside the body cavity of a patient typically requires some form of fixation. There are two commonly recognized forms of lead fixation: passive fixation and active fixation. In passive fixation, flexible tines of silicone or polyurethane typically are used that are designed to engage trabeculae within the right ventricle (RV), for example, in order to secure the lead within the heart. In active fixation, an extendable-retractable metallic helix typically is placed at the distal tip of the lead, which is advanced into the endomyocardium for attachment.

The active fixation leads can be more readily positioned and secured to areas in the ventricle other than the apex, whereas tines tend to more easily find the ventricular apex. Since an implanted device may have a finite life, such as a life of about four years, it can be necessary to remove the device at a later time. Removal of a chronic tined lead can be difficult, however, due to fibrotic ingrowth around the lead tip and tines. Because the tined lead diameter is larger than the more proximal features, the tip typically will resist withdrawal. In contrast, an active fixation helix can be retracted into the tip prior to removal. Further, the tip diameter when using such a helix is the same or smaller than the proximal features. Retraction of the fixation helix requires access to the proximal lead, however, and if the lead is completely intravascular, access to the proximal lead for actuation of a helix is impractical or impossible.

### BRIEF DESCRIPTION OF THE DRAWINGS

FIG. 1 is a side elevation view of a first embodiment of a detachable fixation mechanism.

FIG. 2 is a cross-sectional side elevation view of a second embodiment of a detachable fixation mechanism.

FIG. 3 is a cross-sectional side elevation view of a third embodiment of a detachable fixation mechanism.

FIG. 4A is a side elevation view of a fourth embodiment of a detachable fixation mechanism.

FIG. 4B is a cross-section view taken along the plane designated 4B-4B in FIG. 4A.

FIG. 5 is a side elevation view of a modification to the FIG. 4A embodiment.

FIG. 6 is a side elevation view of a fifth embodiment of a detachable fixation mechanism.

FIGS. 7A-7B are side elevation views of the non-degradable undercut feature of FIG. 6.

FIG. 8A is a side elevation view of a detachable fixation mechanism utilizing electrolytic detachment.

FIG. 8B illustrates the mechanism of FIG. 8A detached from the lead.

FIG. 9A is a cross-section side view having of a detachable fixation mechanism having biodegradable and breakable features.

FIG. 9B and 9C are cross-section views giving two alternatives for the cross-section of FIG. 9A along the plane designated A-A in FIG. 9A.

FIG. 10 is a cross-sectional side view of a biodegradable fixation mechanism.

FIG. 11 is a side elevation view of still another detachable fixation mechanism

FIG. 12 is a side perspective view of a retractable tine assembly.

FIG. 13 is a side perspective view showing the retractable tine assembly of FIG. 12 in a deployed position.

FIGS. 14A-14C are illustrations similar to FIG. 13 showing alternative geometries for retractable tine assemblies.

FIGS. 15 and 16 are perspective views of a retractable tine assembly illustrating methods for retracting the tines.

FIG. 17A is a perspective view of a lead for use with a separately implantable detachable fixation mechanism.

FIGS. 17B and 17C show examples of fixation mechanisms usable with the lead of FIG. 17A.

FIG. 18 is a perspective view of the tip of FIG. 17C being deployed using a tip implantation device having a rotatable bushing.

### DETAILED DESCRIPTION

Systems and methods in accordance with various embodiments of the present invention overcome deficiencies in existing implantable devices by improving upon the mechanisms by which devices are fixed, or anchored, in the body. Implantable devices such as leads for defibrillators can have a fixation mechanism that is at least partially detachable or dissolvable in order to allow for easier removal of the device. These devices can be either actively or passively fixed to tissue, using fixation mechanisms such as removable or dissolvable helices, tines, barbs, or wedges. Such approaches allow the leads to be placed anywhere in the heart (or other appropriate location) while attached to the fixation mechanism, instead of initial placement of a fixation device and then subsequent attachment of the lead as in the prior art.

Further, many existing leads deliver energy for pacing, defibrillation, etc. from the end or tip of the lead. Some embodiments discussed herein do not require energy delivery pacing from the end of the lead, such that a wire does not need to go all the way to the end of the lead. This can be advantageous, as a significant amount of strength is necessary to break such a wire, which can cause injury to the patient (damaging surrounding walls, tissue, etc.) and can leave behind a wire tip that may be difficult to explant. Instead, an electrode or series of electrodes can be used that is more proximal. By breaking off or dissolving the tip, the residual lead can simply be pulled from the body. Other existing devices use retractable screws, but simply pulling withdrawing a screw from the heart muscle can cause significant injury, as discussed.

Mechanical Break

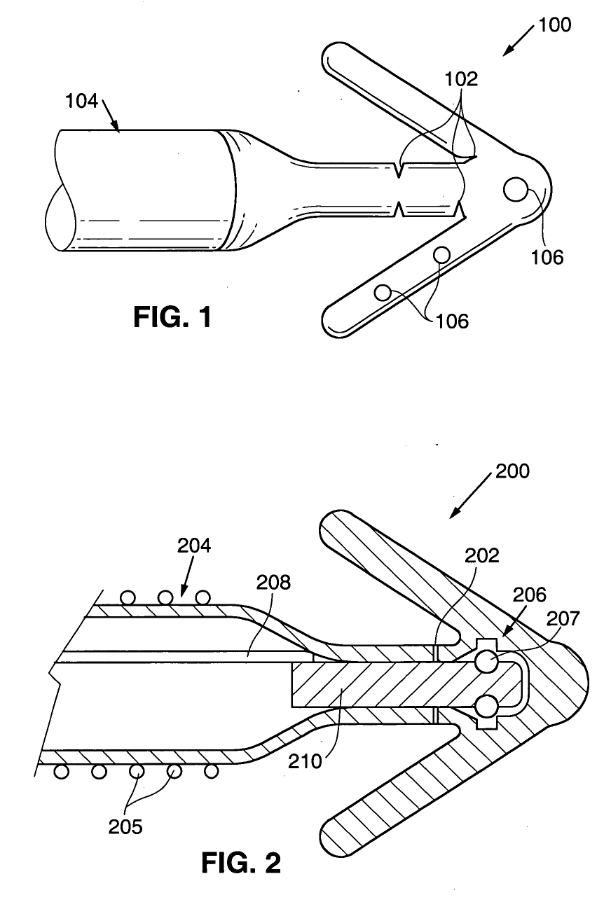

A breakaway fixation mechanism 100 in accordance with a first embodiment, shown in FIG. 1, includes a series of notches 102 formed in a narrow end region of the implantable device (e.g. the lead). The end region is more narrow than the bulk in this device because the electrode only extends to a point 104 that is separated a distance from the end of the fixation mechanism, as opposed to an electrode wire that extends to the end of the fixation mechanism as in previous systems. The number of notches, the size of the notches, and the placement of those notches can depend upon the material being used to form the fixation mechanism 100, the strength needed to anchor the device, and the desired maximum pulling strength that is to be applied in breaking away the fixation mechanism and extracting the remainder of the lead. The fixation mechanism can be any appropriate biocompatible material known in the art for such devices capable of providing necessary anchoring strength. Proper placement of the notches allows such a fixation mechanism to be broken away from a mechanical lead, for example, simply by pulling on the opposite end of the lead while the tines and tip are held by fibrotic tissue. The lead then can be easily extracted with the fixation mechanism, separated at the notches, being left in place. It also can be desirable to include ingrowth retention promoters 106 in the fixation mechanism in order to improve anchoring strength during the first few months after implant. The promoters 106 also provide strength at the time of removal so that the lead can be broken away from the fixation mechanism 100 without damaging the surrounding tissue or becoming partially dislodged. Because the electrode wire does not go to the end of the lead, and therefore does not need to be broken or separated, the strength required to break the device at the notches would be less than for previous devices. Although a tined tip is shown in this example, other fixation mechanisms are possible, such as a helix or screw assembly.

Snap Fit

A breakaway fixation mechanism 200 in accordance with a second embodiment, shown in Fig, 2, includes a slit 202 defining a detachment point between the bulk of the lead 204 (including electrodes 205) and the fixation mechanism 200. Separation at the location of the slit allows the lead to be easily be separated from the fixation mechanism and extracted from the body. A snap fit assembly 206 can be used to hold the fixation mechanism together with the bulk of the lead. The snap fit assembly can include components such as a ball detent 207, an interference fit, an o-ring, and/or a snap-ring. The snap fit assembly allows the fixation mechanism to be easily attached to the end of the lead, with at least one component of the assembly “snapping” into place when the fixation mechanism is attached in order to removably lock the mechanism into place. The snap fit assembly also allows for the easy separation of the fixation mechanism. A cable 208 (preferably inelastic) can be attached as shown, which can apply a load to a post 210, causing a pull out from the fixation mechanism.

In an alternative embodiment, the snap fit may be accomplished using thermal activation using a shape-memory alloy as known in the art. Thermal activation of such an alloy, when used to connect components of the assembly, can deform or otherwise manipulate the shape of the alloy to allow those components to be disconnected. An internal energy source can be used to thermally activate the alloy, or a remote energy source coupled by induction or conduction. Although a tined tip is shown in this example, other fixation mechanisms are possible, such as a helix or screw assembly.

Biodegradable Tip Retainer

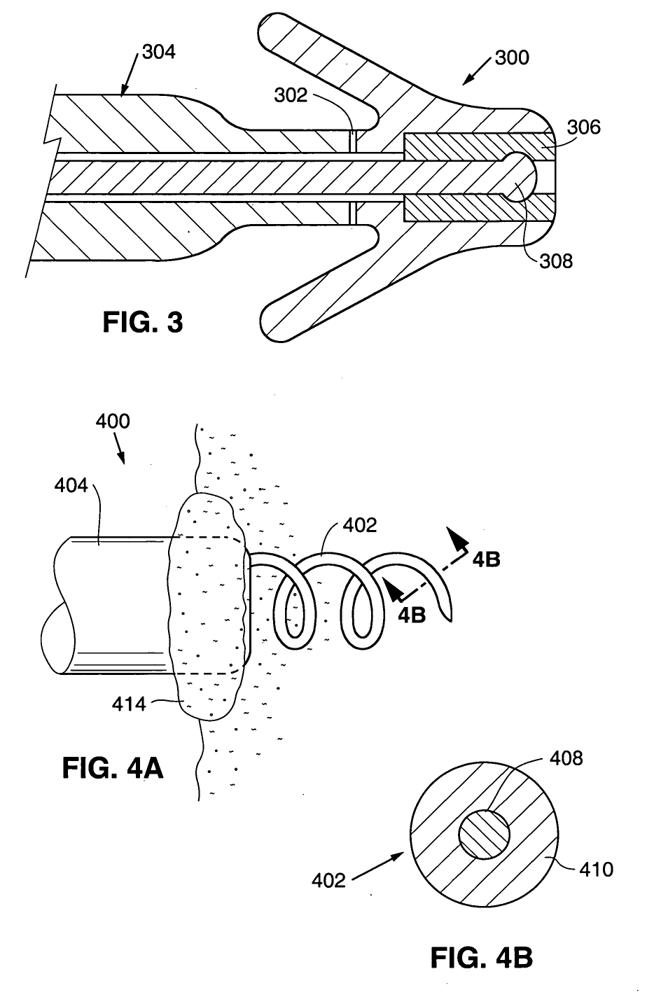

A breakaway fixation mechanism 300 in accordance with a third embodiment, shown in FIG. 3, again utilizes a slit 302 defining a detachment point between the bulk of the lead 304 and the fixation mechanism 300. In this device, however, a biodegradable tip retainer 306 is used in the tip of the lead, in the fixation mechanism 300, to hold the lead in the fixation mechanism. An object can be used at the end of the lead to hold the lead in place in the retainer 306, such as a tether 308 (ball optional) made of cable, wire, polyester yarn, or another porous material. The retainer can be made of any appropriate biodegradable material known in the art and suitable to be implanted in a location such as a right ventricle. Once the retainer material biodegrades, the lead can be pulled to detach the tether 308 from the fixation mechanism 300. The tip can break away or detach at the slit 302, or other detachment point or notch, upon a pulling of the lead.

Helix Coated with a Biodegradable Material

A fixation mechanism 400 in accordance with a fourth embodiment, shown in FIGS. 4A and 4B, includes a helix 402 used to hold the lead 404 in place, such as by being placed into the myocardium of a patient. The helix 402 can consist of an inner helical core 408 and an outer degradable coating 410, such as a polymer or magnesium, as shown in the corresponding cross-section of FIG. 4B. When combined with the coating, the helix 402 can have adequate strength to hold the lead. Over time, the lead can become more stable due to fibrous ingrowth 414 (FIG. 4A), which can be locally promoted using ingrowth-promoting materials or substances on the helix 402. As the coating 410 is resorbed into the body, the resulting helix (composed primarily of the core and any residual coating) can be sufficiently weak to allow safe extraction via traction. In an alternative embodiment shown in FIG. 5, the length of the non-resorbable core 408, which can be a helical wire, for example, can be shorter than the length of the original helix 500, which includes the coating 410, as the core 408 can be tapered and therefore can progressively increase in strength from the distal to the proximal end. In either embodiment, the biodegradation rate can be tailored to match the ingrowth and stabilization rate. As an alternative, the entire helix (or other fixation mechanism) can be biodegradable. Exemplary biodegradable materials/coatings suitable for the various environments include poly caprolactone (PCL), poly glycolic acid (PGA), and poly lactic acids (PLA).

Biodegradable Fixation Mechanism

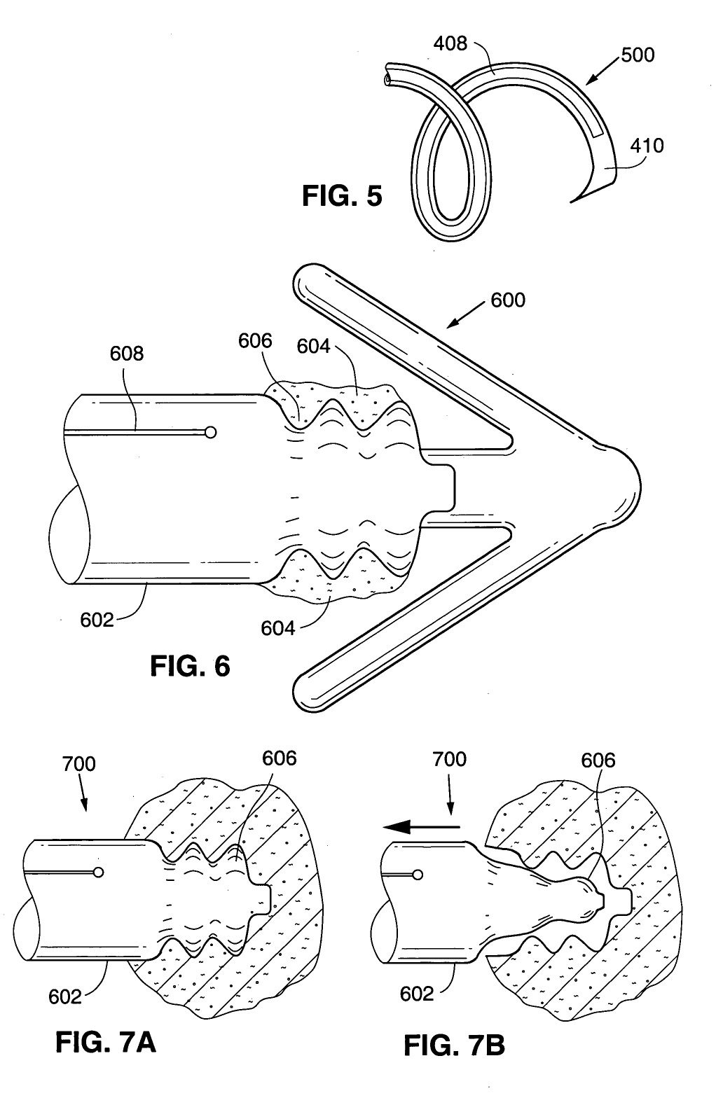

A fixation mechanism 600 in accordance with a fifth embodiment, shown in FIG. 6, is one example of a biodegradable fixation mechanism. The implantable device 602 can be designed to promote fibrous ingrowth, such that after a period of time (such as about 60 days) the ingrowth 604 can be sufficient to hold the lead in place. The fibrous ingrowth can form around a mild undercut feature 606 of the implantable device. An undercut feature forming a transition region can utilize a high elongation material, as known in the art. A retraction wire 608 or cable can terminate just proximal a necking transition. Under a moderate tensile load (traction), the undercut feature 606 can pull away, such as is shown in FIGS. 7A and 7B. Also as seen in FIGS. 7A and 7B, the fixation mechanism has biodegraded and is no longer holding the lead 602 in place. The undercut feature 606 can be at least partially collapsible to a more elongated arrangement as shown, or compressible, upon activation of the retraction wire, such that the feature can be easily extracted without damaging the surrounding tissue. Alternatively, the undercut feature itself can be helical and extraction can be accomplished by twisting the lead (to unscrew the helix).

Electrolytic Detachment

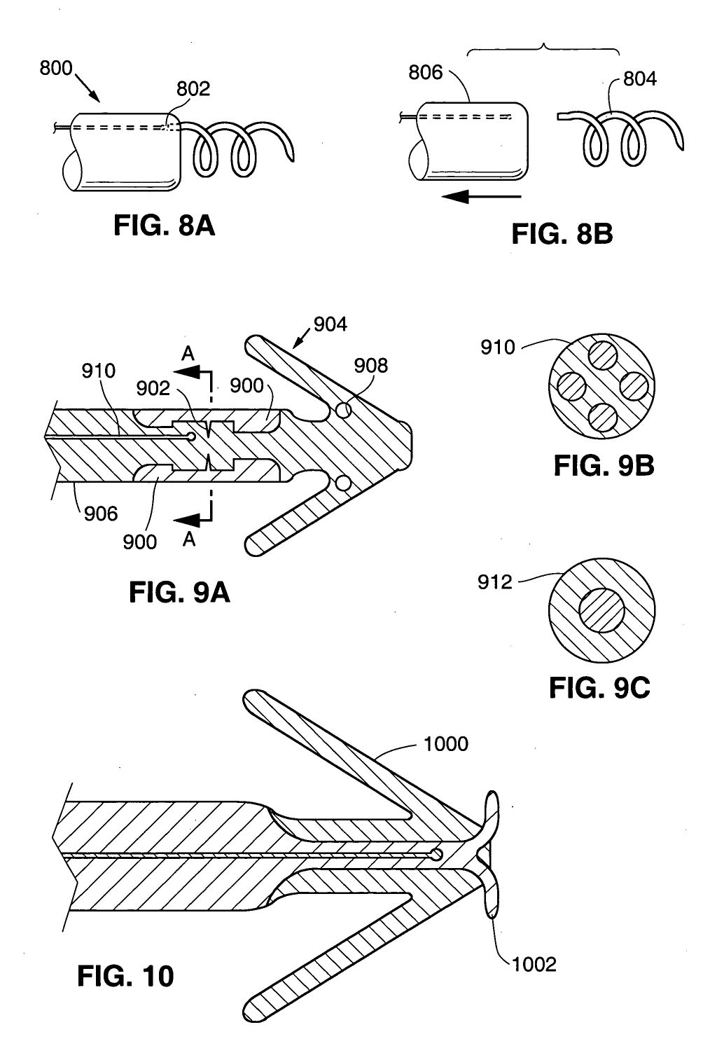

A fixation mechanism 800 in accordance with a sixth embodiment, shown in FIG. 8, includes an electrolytic detachment element 802, which electrolytically erodes when exposed to electrical energy. Element 802 can be energized via a conductor or otherwise through the implanted device. A helix is shown for illustration, but the attachment mechanism could be any mechanism described herein or otherwise useful for anchoring, such as a tine or wedge. The fixation feature alternatively can be caused to straighten or soften through the application of thermal energy to a material such as a shape memory alloy (Nitinol) or polymer, in order to detach the fixation feature. In the example shown, the application of energy causes the helix 804 to detach as shown in FIG. 8B such that the bulk lead 806 can be easily extracted.

Combinations

Other embodiments can combine ideas in the first six embodiments. These concepts could be use independently or in a number of combinations. For example, FIG. 9 shows an embodiment wherein a biodegradable material 900 is positioned about a notch 902 used to allow the tip or fixation mechanism 904 to detach upon extraction of the lead 906. The fixation mechanism can have fibrous ingrowth promoters 908 as discussed above. The biodegradable material can take the form of a biodegradable cuff over the notch, such that when the material degrades (and eventually resorbs), the tensile strength of the lead will be decreased at the notch 902. For example, around the time of implantation (up to 30-90 days), the ultimate strength is about 2.5-3.0 lbs. After the biodegradable material 900 has resorbed, after about 6 months, the tensile strength can be about 0.5-1.0 lbs. or less. Two cross sections 910, 912 are shown in FIG. 9B and 9C, illustrating exemplary shapes of the lead at the notch location surrounded by the biodegradable material. In another embodiment, shown in FIG. 10, a bioresorbable tine 1000 is used as part of the fixation mechanism. These can be used alone or in combination with at least one long-term removable tine 1002. Alternatively, the interface between a permanent tine and lead could be resorbable and/or degradable. This would allow the lead to pull out of the tine at the time of extraction.

Removable Tine

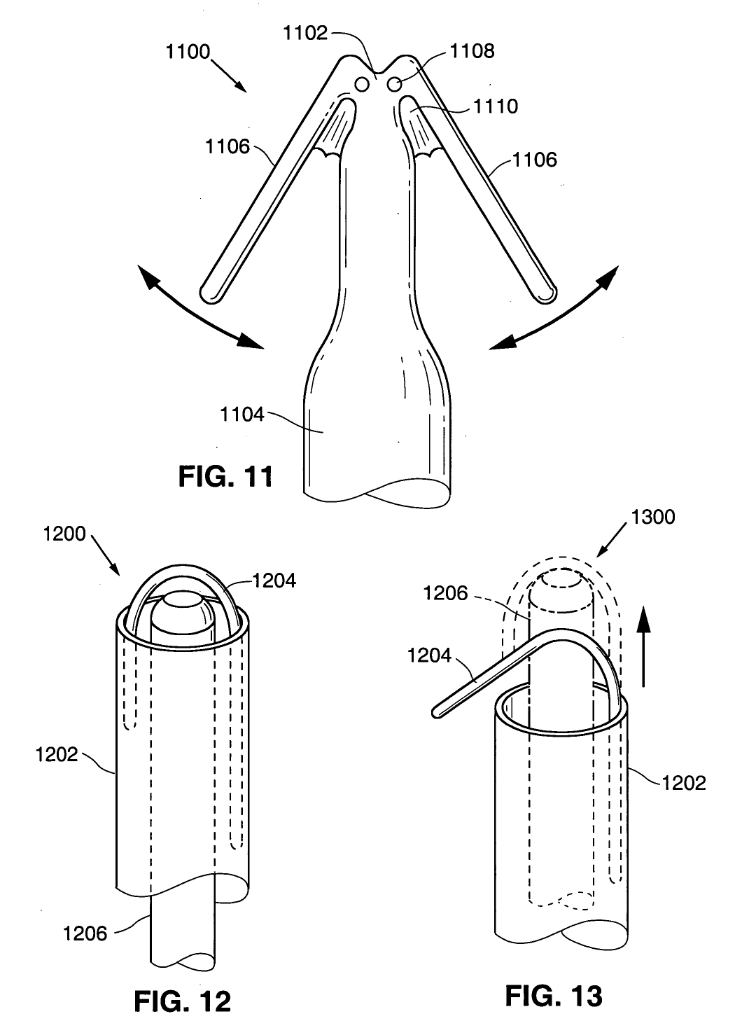

A fixation mechanism 1100 in accordance with a seventh embodiment, shown in FIG. 11, includes a lead tip 1102 that can be removed with the lead 1104, with the tines 1106 being removable. The tip can contain relief 1108 to facilitate straightening of the tine. The location where each tine 1106 attaches to the lead tip 1102 can include a web 1110 that is perforated or notched, such that the web 1106 can be broken off when sufficient traction (such as one pound of force) is applied to the lead and tip. Alternatively, a biodegradable polymer can be used that would dissolve and/or resorb or weaken after the lead tip is held by in-growth of tissue. The weakened tines can then be prolapsed or inverted allowing withdrawal of the tines 1106 with the lead 1104.

Extendable/Retractable Tine Anchors

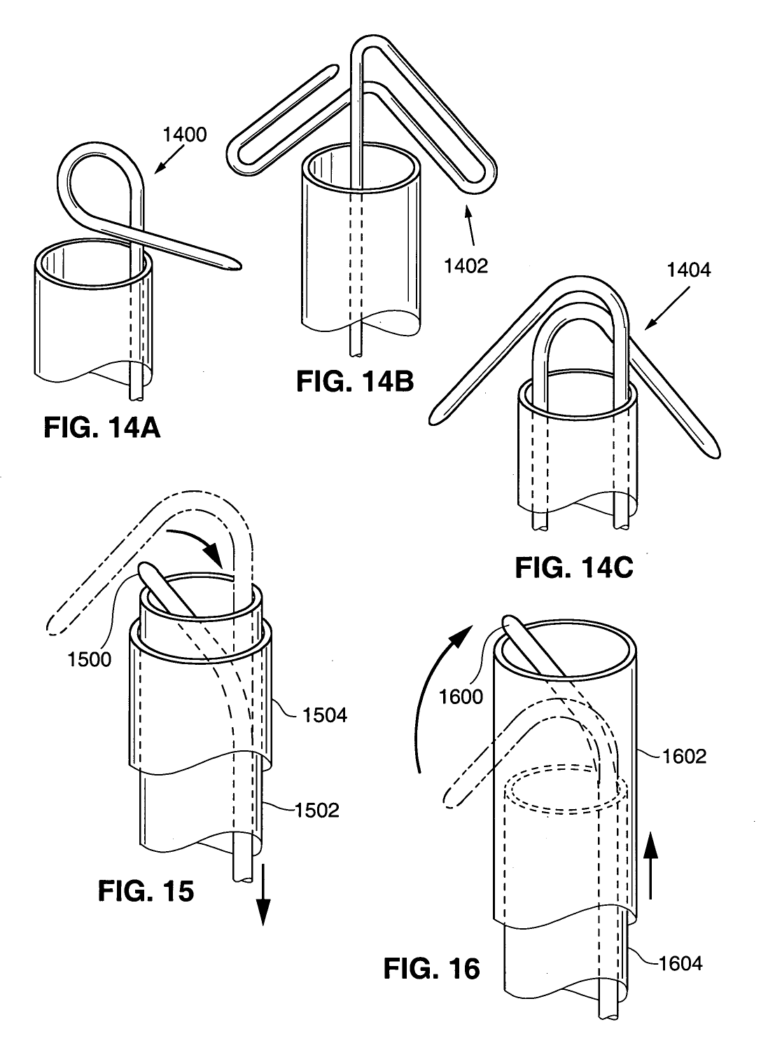

A fixation mechanism 1200 in accordance with an eighth embodiment, shown in FIG. 12, allows a lead 1202 to be delivered (such as into the right ventricle (RV)) with the tine(s) 1204 retracted. In this design, the retracted tines do not extend out past the circumference of the lead. The retracted tine(s) can be constrained within the tip of the lead. The tine(s) 1204 then can be advanced to an extended position forming a fixation device 1300 once the lead is in place, such as is shown in FIG. 13. Each tine can be constructed from a material such as nitinol wire, for example, with or without a coating. A stylet 1206 can be used that facilitates delivery, and that can be used as a plunger to expose the tines when advanced to anchor the lead in place. FIGS. 14A-C show some possible alternative tine geometries 1400, 1402, 1404 that can be retracted and advanced. FIG. 15 shows a view of a tine 1500 being retracted in order to remove the lead 1502. The tine wires can be pulled from the proximal end, possibly with counter-traction at the lead tip. A counter-traction sheath also can be used to facilitate retraction of the tine(s). Alternatively, as shown in FIG. 16, the tine(s) 1600 can be withdrawn into a sheath 1602 that is advanced over the lead 1604. If the lead body has a small amount of axial elasticity, and the tine 1600 is anchored proximally (having a minimal amount of stretch), the traction force can pull the tine(s) into the tip.

Fixation Plugs

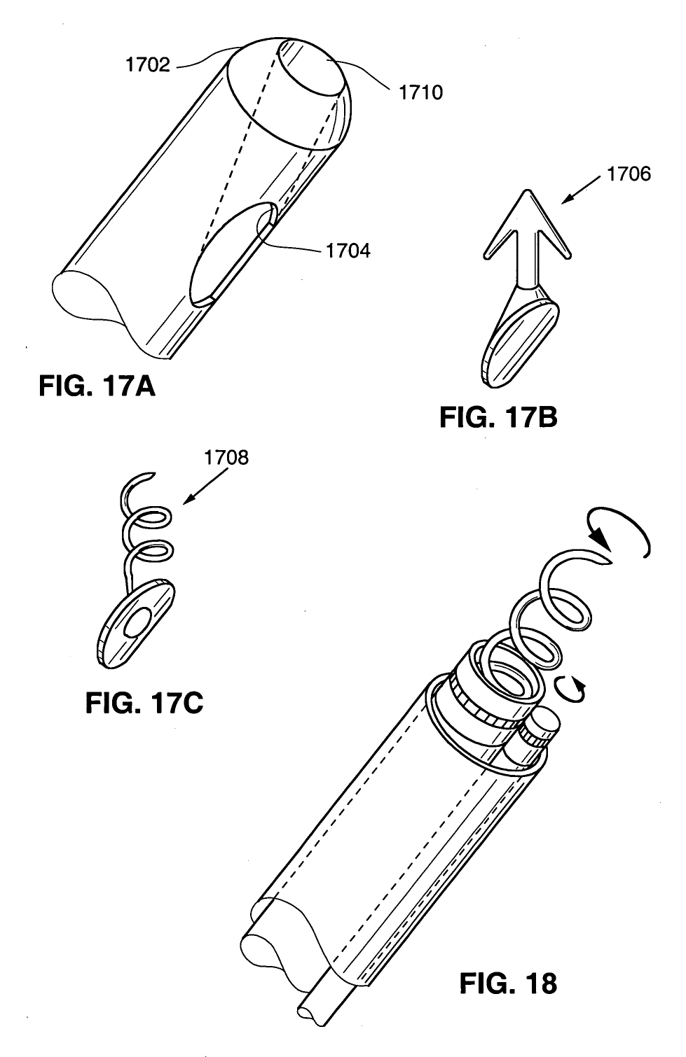

Alternatively, a fixation mechanism can include a fixation plug capable of being delivered independently by a lead delivery system. Such a feature can be biodegradable, facilitating removal of the lead. In one such device, the tip 1702 of the lead 1700 (FIG. 17A) can have an opening 1704 shaped to receive a fixation device such as a barb 1706 (FIG. 17B), staple, helix 1708 (FIG. 17C), or screw. With the lead 1700 at an implantation site, the fixation device can be inserted into the opening 1704 and pushed into position where the fixation device extends out a second opening 1710 at the end of the tip 1702 for holding the lead in place. As shown in FIG. 18, there can be a geared bushing 1800 or externally actuated screw in the lead for externally actuating a screw or helix fixation device requiring rotation for insertion into the tissue. The bushing can be driven using a motor or manual means. Alternatively, the fixation device may be implanted prior to the lead, an din a later step the lead may be advanced such that its distal opening 1710 passes over the fixation device until the two elements are engaged. In either method, tension is applied to the lead to detach the lead from the fixation device.

Although the embodiments disclosed herein are described in the context of leads fixed in the heart, it should be appreciated that the disclosed principles are applicable to other types of implantable devices as well. For example, intravascular devices, including those of the type disclosed in U.S. Pat. No. 7,082,336 and U.S. patent application Ser. No. 10/862,113, owned by the assignee of the present application, include radially expandable anchors expandable into contact with the wall of a blood vessel and the implantation site. Detachment mechanisms of the type disclosed herein may be employed to allow separation of the intravascular device (e.g. pulse generator or vascular lead) from the anchor without causing trauma to the vessel wall.

It should be recognized that a number of variations of the above-identified embodiments will be obvious to one of ordinary skill in the art in view of the foregoing description. Accordingly, the invention is not to be limited by those specific embodiments and methods of the present invention shown and described herein. Rather, the scope of the invention is to be defined by the following claims and their equivalents.

Any and all patents, patent applications and printed publications referred to above, including those relied upon herein for purposes of priority, are fully incorporated by reference.

What is claimed is:

1. A cardiac pacing or defibrillation lead, comprising:
an elongated lead body including a separation point defining distal and proximal portions of the lead body;
at least one exposed electrode in the proximal portion of the lead body near the separation point; and
a non-conductive distal fixation component connected to the distal portion of the lead body, such that when the elongated lead body is separated at the separation point the fixation component retains the distal portion in position and allows the proximal portion with the at least one exposed electrode to be extracted.

2. A lead according to claim 1, wherein:
the proximal and distal portions are separated by breaking the lead body at the separation point under application of traction.

3. A lead according to claim 1, wherein:
the fixation component and a portion of the lead body are coupled by a coupling releasable upon the application of traction exceeding a predetermined force.

4. A lead according to claim 1, wherein the coupling includes a coupling selected from the group consisting of ball-detents, interference fit, and snap-fit couplings.

5. A lead according to claim 4, wherein at least a portion of the coupling includes a biodegradable component.

6. A lead according to claim 1, wherein:
the distal portion of the lead body includes in-growth promoters.

7. A lead according to claim 6, wherein:
the in-growth promoters are selected from the group consisting of undercuts, holes, fibrous materials, porous materials, and biologically active materials.

8. A cardiac pacing or defibrillation lead, comprising:
an elongated lead body having a proximal end and a distal end;
at least one exposed electrode near the distal end of the lead body; and
a detachable fixation component connected near the distal end of the lead body, such that when the fixation component is detached from the lead body the lead body and exposed electrode can be extracted.

9. A lead according to claim 8, wherein:
at least a portion of the fixation component is biodegradable.

10. A lead according to claim 8, wherein:
the fixation component is detachable from the lead body by electrolytic detachment.

11. A lead according to claim 8, wherein:
the fixation component is detachable from the lead body upon application of a traction force, and wherein the traction force needed to achieve detachment decreases over time.

12. A lead according to claim 8, wherein:
the fixation component includes in-growth promoters.

13. A lead according to claim 12, wherein:
the in-growth promoters are selected from the group consisting of undercuts, holes, fibrous materials, porous materials, and biologically active materials.

14. A lead according to claim 8, wherein:
the detachable fixation component includes a degradable first portion overlaying a more flexible non-degradable second portion.

15. A lead according to claim 8, wherein:
the detachable fixation component is a resorbable tine.

16. A lead according to claim 8, wherein:
the detachable fixation component is a resorbable helix.

17. A lead according to claim 8, wherein:
the detachable fixation component is at least partially resorbable.

18. A lead according to claim 8, wherein:
the detachable fixation component is extractable.

19. A lead according to claim 18, wherein:
the detachable fixation component is selected from the group consisting of webbed tines and coated screws.

20. A method of removing a lead from a body, comprising the steps of:
providing a lead including an elongated lead body having a proximal end and a distal end, at least one exposed electrode near the distal end of the lead body, and a detachable fixation component connected near the distal end of the lead body, with the lead implanted in a human body with the fixation component engaged with a portion of the body, applying traction to a portion of the lead body, causing the lead body to separate from the fixation component and extracting the lead body and exposed electrode from the human body.

21. The method of claim 20, wherein the applying step causes the lead body and fixation component to break apart at a predefined separation point.

22. The method of claim 20, wherein the providing step provides the lead body and fixation component to be coupled by a coupling releasable upon the application of traction exceeding a predetermined force.

23. The method of claim 22, wherein the method further includes the step of, prior to the causing step, allowing a portion of the coupling to biodegrade or bioresorb.

24. The method of claim 20, wherein the method further comprises, after the extracting step, allowing the fixation component to biodegrade or bioresorb within the body.
